(19) United States
(12) Patent Application Publication
(10) Pub. No.: US 2005/0260183 A1
(43) Pub. Date: Nov. 24, 2005

(54) Treatment of surfaces populated by bacteria with a lucilia sericata extract

(76) Inventor: David Idris Pritchard, Leicester (GB)

Correspondence Address: JOHN S. PRATT, ESQ;KILPATRICK STOCKTON, LLP, 1100 PEACHTREE STREET, ATLANTA, GA, 30309, US

(21) Appl. No.: 10/506,948

(22) PCT Filed: Mar. 6, 2003
(86) PCT No.: PCT/GB03/00959

§ 371 Date: Apr. 29, 2005

(30) Foreign Application Priority Data
Mar. 9, 2002 (GB) 0205593.7

(51) Int. Cl. A61K 38/46; A61K 31/65

(52) U.S. Cl. 424/94.6; 424/423; 514/153

(57) ABSTRACT

A surface populated by a bacteria capable of producing a biofilm is treated beneficially by contacting it with a substance having N-acyl homoserine lactone degradant activity obtained from the secretions and/or excretions of the larval form of the green bottle fly, Lucilia sericata. Furthermore, a synergistic effect is achieved when the larval secretions/excretions are used in combination with an antibiotic, e.g. tetracycline. Such a substance having N-acyl homoserine lactone degradant activity obtained from Lucilia sericata larval secretions and/or excretions also forms part of the invention.

The present invention relates to a method of treating a surface populated by bacteria capable of producing a biofilm using secretions of Lucilia sericata larvae and to compositions useful in such a method.

Biofilms are biological films which develop and persist at surfaces. They may be found on the surfaces of industrial equipment where liquids are transported or processed, in plumbing systems, and on surfaces adjacent to such equipment or systems. They are often found on the surfaces of medical implants or devices inserted into the body. They may also form in areas of the body which are open to the air; in particular they may be found in wounds and on the lining of the lungs. A biofilm can be described as a bacterial population enclosed within a polysaccharide matrix which adheres to surfaces.

Biofilms are generally stable formations which are difficult to treat by conventional techniques. This is due to the protective nature of a polysaccharide biofilm matrix in which the micro-organisms are embedded. Conventional medicaments, such as antibiotics, are less effective either due to diffusion barriers or the altered metabolic state of micro-organisms in the biofilm.

Biofilm formation is thought to involve the production, by the micro-organisms, of diffusible signal molecules by a process known as quorum sensing. These molecules are thought to trigger production, by the micro-organisms, of exo-polysaccharides, exo-proteins and other secondary metabolites. Compounds which interfere with these molecular processes may inhibit biofilm formation and/or weaken an already formed biofilm.

Pseudomonas aeruginosa is one of the most common and problematical of infective bacteria. It is particularly problematical in that it forms biofilms which are difficult to treat with conventional antibiotics. Biofilm formation by Pseudomonas aeruginosa is problematical for patients with cystic fibrosis in whom it colonises the lungs causing infections which are difficult to treat and often ultimately fatal.

Efficient wound healing is a complex physiological process which involves many mechanisms including cell migration, growth factor secretion, angiogenesis, tissue remodelling and the intrinsic proteinase/antiproteinase balance of the wound contributing in concert and in an apparently staged manner to accelerate controlled tissue regeneration.

Wound care products are essential in modern medical practice, especially for the treatment of patients with chronic wounds or burns. Many different substances have previously been proposed as having activities which contribute to the healing of wounds. These previously proposed substances include streptokinase, collagenase and streptodornase (all obtained from bacterial sources), bromelain (from pineapples), plasmin and trypsin (obtained from cattle) and krill enzymes (obtained from crustacea). Clinical trial data indicate that such substances are only partially effective in promoting the healing of wounds.

The larvae (maggots) of the green bottle fly, Lucilia sericata, are known to have significant wound healing attributes as live organisms. Debridement treatment using the larvae of Lucilia sericata has become a widely accepted clinical practice. However, little has been reported in the literature about the way in which these larvae go about their task of cleaning wounds to an extent that conventionally intransigent- wounds heal. Healing can be mechanical, biochemical or a combination of both. Our work shows that the effects of these larvae can be mimicked using extra-corporeal secretions.

Although efficacious, live larvae are unpleasant to many patients and the use of live larvae on wounds and the introduction of their crude secretions into wounds, which inevitably occurs when the larvae are used, are unacceptable to many patients and to many medical practitioners. The use of live organisms also increases the risk of allergic reactions in the patient.

The excretions/secretions (ES) of Lucilia sericata larvae are known to contain an enzyme which exhibits trypsin-like serine proteinase activity. This invention is based on the discovery that extra-corporeal ES also have the ability to break down the low molecular weight signalling molecules produced by bacteria to determine the density of the bacterial population and, thus, disrupt the bacterial messaging network on which biofilm formation depends.

The present invention provides, in a first aspect, a method of treating a surface populated by a bacteria capable of producing a biofilm which comprises contacting the surface with a substance having N-acyl homoserine lactone degradant activity obtained from the secretions/excretions of Lucilia sericata. Typically, the bacteria capable of producing a biofilm is Pseudomonas aeruginosa or Staphylococcus aureus.

It is now becoming more generally accepted that the study of biofilm is more closely allied to the natural patterns of existence of bacteria than the more widely studied planktonic systems. Thomas S. et al, J. Tissue Viability, 1999 Vol 9 No.4 p127-132 reported antimicrobial activity of secretions of Lucilia sericata larvae on planktonic bacterial cells. We have not been able to reproduce these results. However, we have, in our experiments, found that under laboratory conditions the secretions of Lucilia sericata larvae have activity which prevents or reduces the formation of bacterial biofilms. In a wound situation the removal of bacterial biofilm would be advantageous in combating infection.

Our experiments have shown that exo-polysaccharides, which are formed as part of a bacterial biofilm, are removed by the action of the Lucilia sericata secretions. This effect is persuasive of the presence, in the secretions, of glycosidase activity.

The healing of a chronic wound has been shown to be impaired by the presence of a bacterial infection. The level of infection affects the balance between healing and chronicity. The bacterial contribution to wound hypoxia and pathological effects are an impediment to efficient healing.

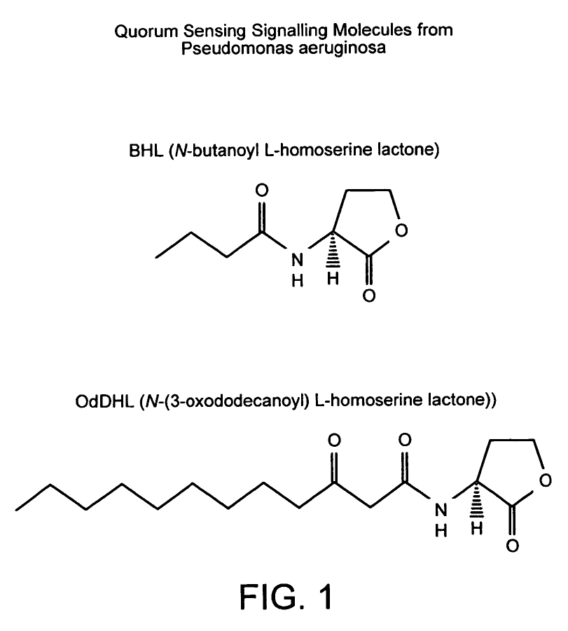

It is known that diffusible low molecular weight signalling molecules allow individual bacteria to determine population density and thus to act in a concerted way when a “quorum” is assembled. This effect, generally known as “quorum sensing”, is now known to control the production of virulence determinants in pathogens, thus ensuring a consolidated attack at a critical stage of infection. The low molecular weight signalling molecules (quorum sensing molecules) are known to include N-acyl homoserine lactones, e.g. N-butanoyl L-homoserine lactone (BHL) and N-(3-oxododecanoyl)-L-homoserine lactone (OdDHL) from Pseudomonas aeruginosa (see FIG. 1). Lactones also cause apoptosis in eukaryotic tissues therefore acting in opposition to the tissue regeneration required for wound healing. It is clear that the breakdown of such signalling molecules would reduce the ability of bacteria to mount an effective attack on host tissues. By our experiments we have shown that the secretions of Lucilia sericata larvae contain an activity which degrades BHL and, to a lesser extent, OdDHL. BHL is thought to be more active once bacteria form a biofilm. This activity has been found by us to be heat stable yet sensitive to phenylmethanesulphonyl fluoride (PMSF) and to 4-amidophenyl-methanesulphonyl fluoride (APMSF), both of which are known to inhibit serine protease and esterase activity.

The secretions from Lucilia sericata larvae may be collected by washing sterile larvae with phosphate buffered saline followed by filtering, under sterile conditions.

We have surprisingly found that the larval secretions when used in combination with a conventional antibiotic, such as tetracycline, have an antimicrobial effect which is greater than the individual effects of the larval secretions and antibiotic when used separately. There is, therefore, some synergy between these individual substances when used in combination. Thus, according to a further aspect, the present invention provides an antimicrobial composition comprising the secretions of Lucilia sericata larvae and one or more antibiotic compound. In a preferred embodiment, the antibiotic compound is tetracycline.

During the course of our investigations, we realised that many important components of larval secretions could be overlooked, as they might be inducible in haemolymph or other tissues by exposure to bacteria (as described by Hoffmann et al (1999) in Drosophila melanogaster) rather than constitutively expressed. Consequently, we have examined phenotypically-altered L. sericata larvae after exposure to bacteria in vitro for the production of antimicrobial compounds in the haemolymph.

The sterile secretions with or without the addition of a conventional antibiotic may be delivered onto a wound area using any known dermal delivery system or may be incorporated into a sterile support, such as a poultice, to be applied to a wound area as a dressing.

### EXPERIMENTAL

Method for Preparation of L. sericata Secretions

Day old (1^{st} instar) larvae of the greenbottle Lucilia sericata were purchased from the Surgical Materials Testing Laboratory (SMTL) Bridgend. These were raised under sterile conditions and all operations to collect the secretions were carried out using sterile equipment and in a laminar flow cabinet. Secretions were collected following the addition of 200 μl phosphate buffered saline (PBS) to each universal (containing approx 200 larvae). The larvae were washed in the PBS for 20 min before the removal (sterile pipette), centrifugation (13,000×g for 10 min) and freezing (−20° C.) of the secretions. The larvae were allowed to rest for 40 min before a second wash and the process repeated for a third wash.

The collected secretions were assayed for protein content (BioRad protein assay) and protease activity (hydrolysis of fluorescein isothiocyanate labelled (FITC)-casein). The secretions were sterile filtered (22 μm filter) and aliquotted ready for use and stored at −20° C.

1. Synergistic Action of L. sericata ES with Conventional Antibiotics

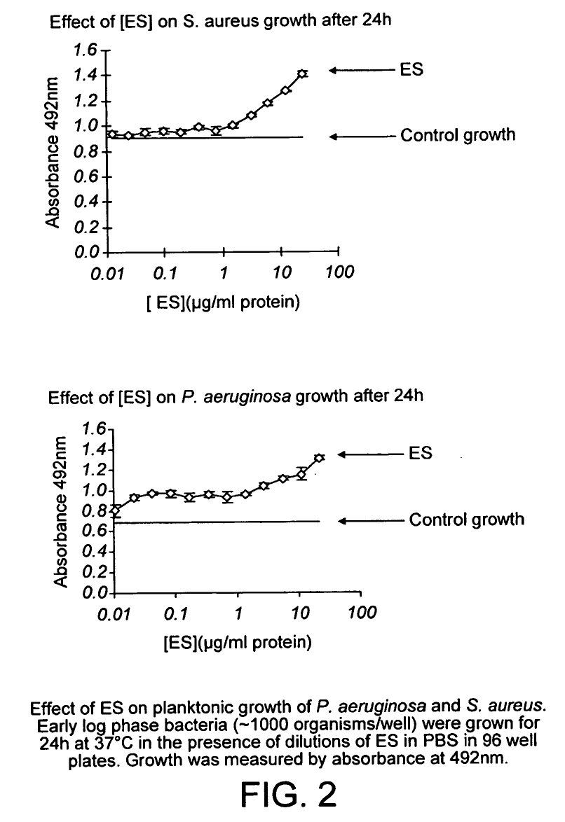

Minimum inhibitory concentration assays were used to determine the concentration of ES that would inhibit growth of planktonic bacteria in a 96 well plate—the growth being measured by increase in absorbance at 492 nm. In contrast with the observations of Thomas et al. (1999) the results showed that ES produced from L. sericata grown under sterile conditions had no antibiotic effect on growth of either gram+ve or gram−ve bacteria (S. aureus and P. aeruginosa respectively) (FIG. 2).

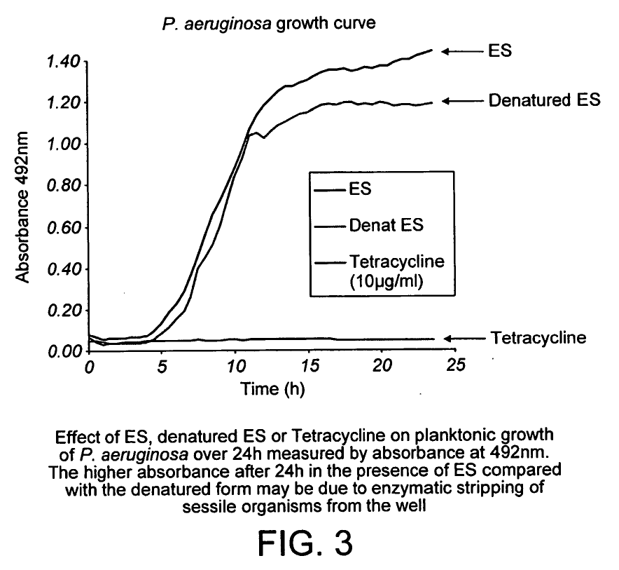

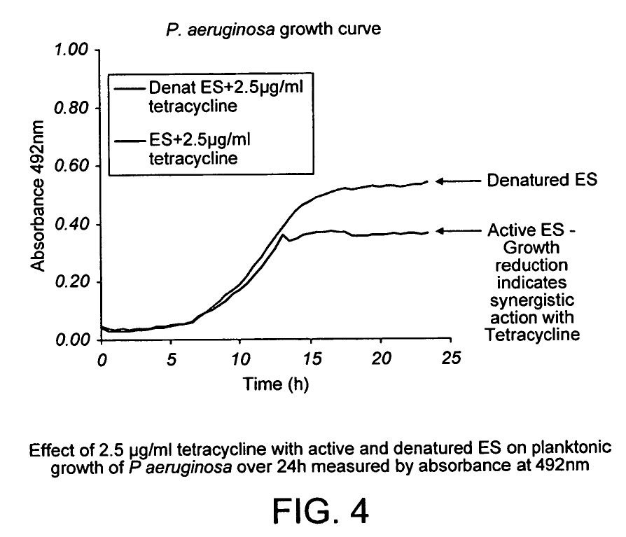

Similarly, following absorbance at 492 nm over a 24h period, the effect of ES and heat-denatured ES (boiled for 10 min) was compared with the effect of the conventional antibiotic tetracycline on growth of P. aeruginosa (FIG. 3). Growth was completely abolished by 10 μg/ml tetracycline. By contrast the active ES showed a slight enhancement of growth (possibly due to biofilm stripping—see below FIG. 4) measured by this method. However, using a sub-lethal level of tetracycline in combination with ES, there was found to be a synergistic effect between the two resulting in lower growth of the bacteria (FIG. 4). This has also been confirmed using colony counts after 24h where the combination of tetracycline and active ES was found to reduce viable counts by one third.

Conclusion—L. sericata secretions alone did not have an antibiotic effect on bacterial growth under planktonic conditions, however there was a synergistic effect with the conventional antibiotic tetracycline.

2. Reduction in Biofilm Formation in the Presence of L. sericata ES

It is now becoming more generally accepted that the study of biofilm is more closely allied to the natural patterns of existence of bacteria than the more widely used but inappropriate planktonic systems. In the wound situation the removal of bacterial biofilm would be advantageous in combating infection.

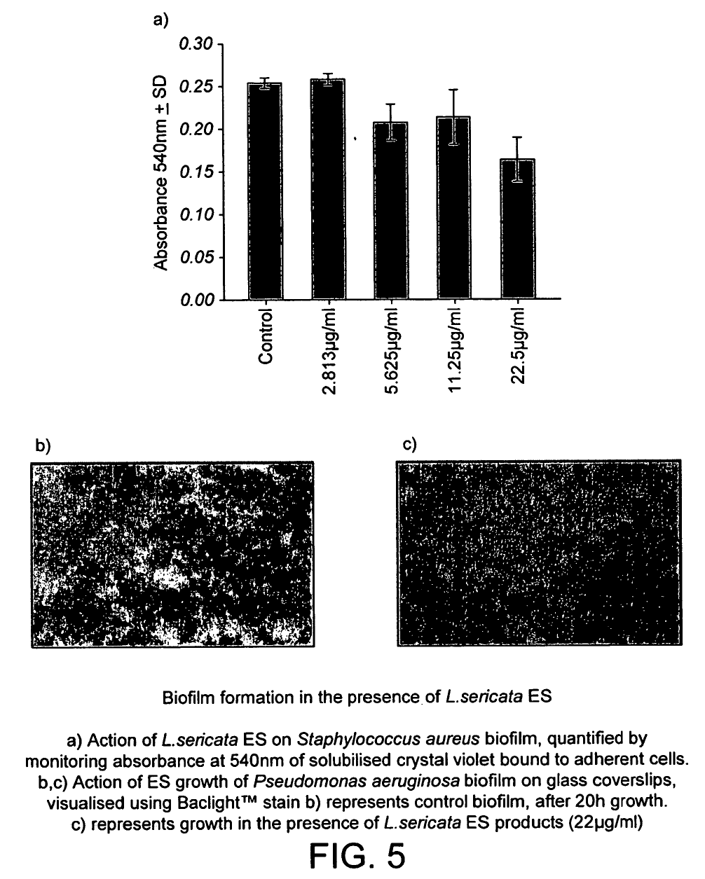

Reduction of biofilm formation in the presence of L. sericata ES was measured using the crystal violet method of O'Toole and Kolter (1999). Attached bacteria were quantified after 24h growth of culture in 96 well plates following removal of the planktonic bacteria (see section 3). Crystal violet dye was used to stain the attached bacteria before solubilisation and measurement of the absorbance of the stain at 540 nm. A dose response reduction in biofilm formed was measured for S. aureus biofilm over 24h in the presence of L. sericata ES (FIG. 5a). A similar reduction in P. aeruginosa biofilm formation was seen after incubation of glass coverslips in cultures grown in the presence of ES. Micro-colonies were visualised using Baclight stain (FIG. 5b and 5c).

Conclusion—L. sericata secretions were active in preventing formation of bacterial film under laboratory conditions.

3. Glycosidase Activity of L. sericata ES Products

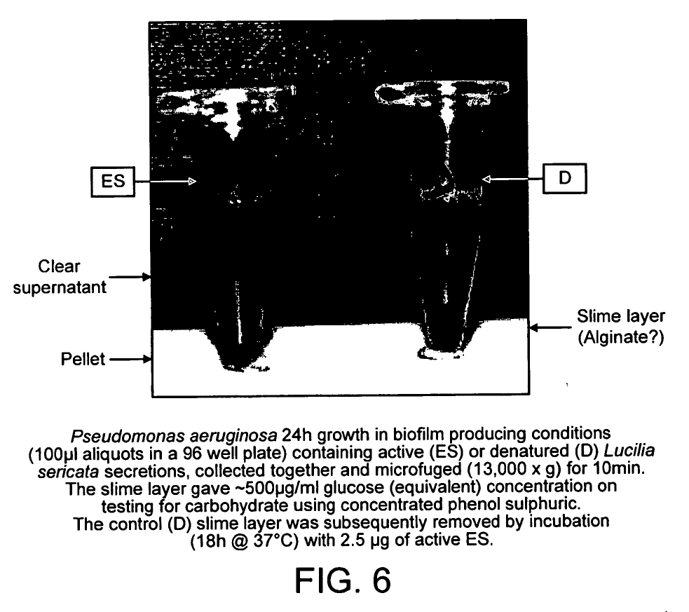

The recovery of cells from cultures grown under biofilm producing conditions revealed differences when the cultures were grown in the presence of L. sericata ES products. The culture was grown in 100 μl aliquots in a 96 well microtitre plate. This has the effect of increasing the surface area of liquid in contact with the plastic well surface in comparison with flask grown culture, thus promoting the growth of biofilm. P. aeruginosa was inoculated from an overnight culture and grown to early exponential phase before dilution (1/2000) to give ˜10³ cells per well. The culture was then grown in the presence of ES, inactivated ES (boiled 10 min) or phosphate buffered saline (control). The culture was grown overnight at 37° C. before collection together of each type of aliquot and centrifugation (13,000×g for 10 min) to recover the cells. The results indicated (FIG. 6) the presence of a “slime layer” in the control and denatured ES samples that was absent when cells were grown in the presence of active ES. The addition of an aliquot of active ES to sample D (denatured ES) followed by incubation overnight at 37° C. resulted in the removal of the slime layer originally formed. We propose that the slime layer may consist of exo-polysaccharide formed as part of the biofilm and removed by the action of glycosidase in L. sericata ES. In the case of P. aeruginosa the exo-polysaccharide has been suggested to be alginate (a polymer consisting of poly guluronic and mannuronic acids).

Conclusion—The enzymatic removal of the exo-polysaccharide would compromise the ability of the bacteria to form an effective biofilm and therefore to establish an effective infection in the wound.

4. Inactivation of P. aeruginosa Quorum Sensing Signalling Molecules by a Thermostable PMSF/APMSF-Sensitive Activity (lactonase) from L. sericata Excretory/Secretory Products (ES)

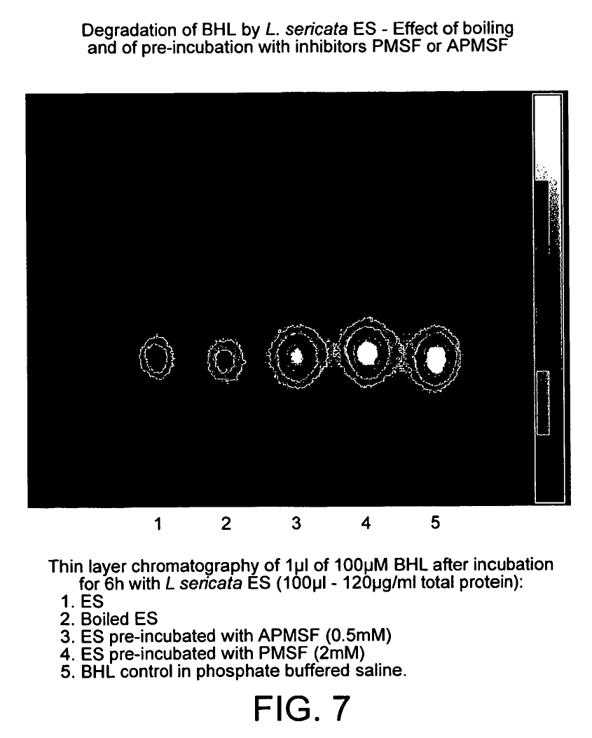

BHL and OdDHL may be quantified using thin layer chromatography (TLC) (RP18 F₂₄₅S or RP2 UV₂₅₄ plates respectively). After chromatography the position and amount of the signalling molecule may be revealed by their effect on biosensor organisms. The particular organisms used emit light when in contact with BHL or OdDHL. Therefore if the TLC plate is overlaid with soft agar containing the biosensor organism the position of the signalling molecule will be revealed by emission of light after a period of incubation. The intensity of the light emitted here is shown by converting to pseudo colour in which the most intense light shows as yellow with a gradation to the least intense—dark blue (FIG. 7—side bar).

The effect of L. sericata secretions on BHL was examined by incubating the following:
1. 100 μl ES (120 μg/ml protein)+100 μM BHL
2. 100 μl boiled ES (120 μg/ml protein)+100 μM BHL
3. 100 μl ES (120 μg/ml protein) pre-incubated with APMSF (0.5 mM)+100 μM BHL
4. 100 μl ES (120 μg/ml protein) pre-incubated with PMSF (2 mM)+100 μM BHL
5. 100 μM BHL in buffer (phosphate buffered saline)

After incubation for 6 hours, 1 μl from each incubation was applied to an RP18F_{254S} TLC plate and separated using 60%(v/v) methanol/water, before using the biosensor overlay to reveal the BHL as described above.

The results (FIG. 7) demonstrate the effect of larval ES on degradation of BHL. The positive control (lane 5) showed light production from the BHL alone. There was a decrease in light production with BHL incubated in the presence of larval ES (lane 1) indicating degradation of the signalling molecule. This degradation was prevented by pre-incubation of the ES with phenylmethanesulphonyl fluoride (PMSF)(lane 4) and to a lesser extent 4-amidinophenyl-methanesulphonyl fluoride (APMSF)(lane 3)(inhibitors of serine protease activity). Boiling of the ES (lane 2) did not prevent degradation thus indicating thermal stability of the activity.

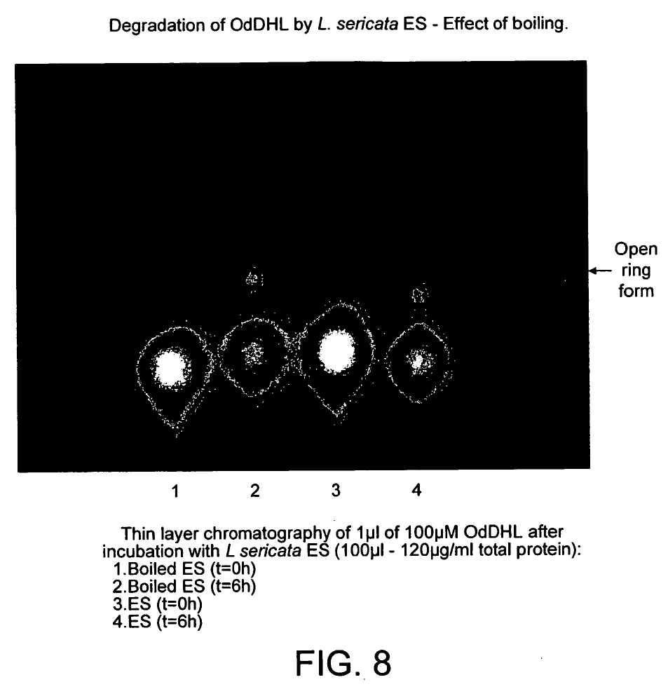

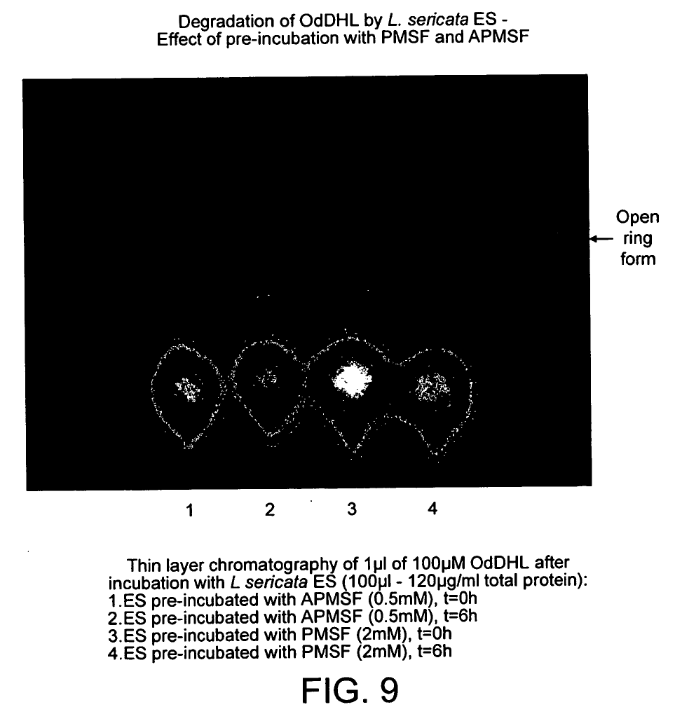

Further experiments confirmed a similar effect of ES on OdDHL (FIGS. 8 and 9) comparing samples taken at the start and end of a 6-hour incubation. This time the TLC was carried out on RP2/UV₂₅₄ plates and with 45% (v/v) methanol/water. The degradation resulted in the appearance of a second species believed to signify ring opening of the OdDHL molecule. This remains to be confirmed by column chromatography (high performance liquid chromatography on a C18 column). Once again the degradation was stable to boiling but inhibited by PMSF and to a lesser extent APMSF.

Conclusion—L. sericata larval ES contains a thermostable activity sensitive to PMSF and APMSF which degrades BHL and OdDHL, two signalling molecules involved in P. aeruginosa biofilm formation and infection and therefore with implications for wound healing.

5. Induced Anti-Microbial Activity in L. sericata

Sterile larvae of L. sericata were obtained from Surgical Materials Testing Laboratory SMTL (Princess of Wales Hospital, Bridgend CF31 1 RQ). The larvae were grown on medium described by Sherman (1995), comprising decomposed pig's liver and bacto-agar, sterilised by autoclaving in a closed container which allowed the exchange of gas and moisture between the interior and exterior of the container but which prevented the entry, into the container, of bacteria. A thin layer of nutrient medium, for the larvae, was provided in the base of the container.

Sterile first instar larvae (200) were suspended in 200 μl sterile phosphate buffered saline and transferred to the container. Growth was allowed under sterile conditions in a moisture chamber at 28° C. for ˜48h to allow establishment of the larvae. Pseudomonas aeruginosa, mutant PAO P47, was inoculated into 10 ml Luria Bertani (LB) medium and grown overnight with shaking at 37° C. The container was inoculated with 1 ml (˜10⁸ viable counts) of the culture and the larvae allowed to grow in the presence of the bacteria. After a further 48h the larvae were sacrificed and haemolymph collected after dorsal anterior incision. The haemolymph was microfuged (13,000×g for 10 min) to remove cells.

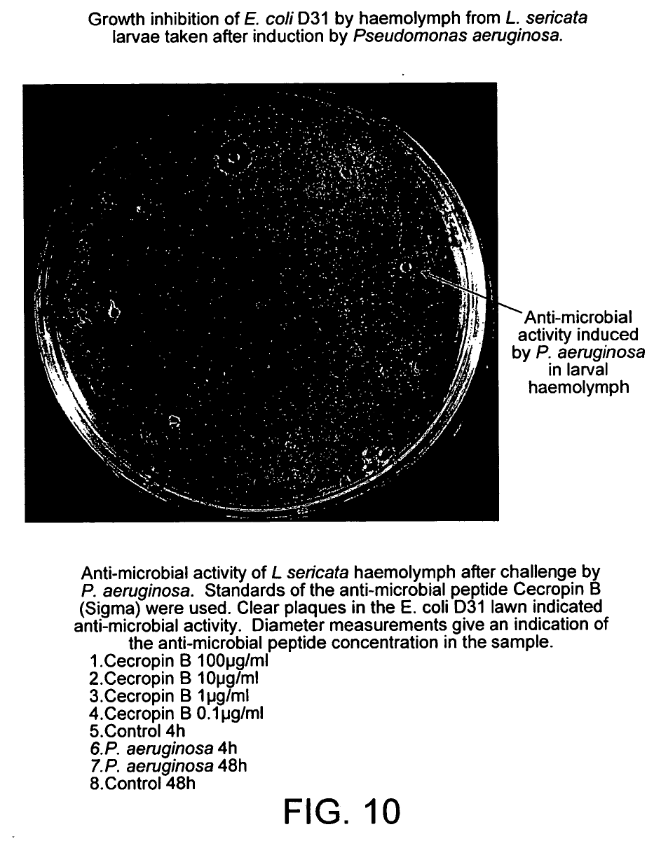

Anti-microbial activity was assessed by the formation of bacteria-free plaques around wells containing 2 μl haemolymph in a bacterial lawn of E. coli D31. This plate was prepared by inoculating 1 μl of culture (Absorbance 600 nm=0.4) into 7 ml of molten (50° C.) 1% LB agar containing 10 μg/ml streptomycin and 5 mg/ml lysozyme. This was formed in a sterile petri dish. The wells (8) were formed in a regular pattern equidistant from the edge of the plate using a template. The anti-microbial activity was assessed by comparison with plaques produced by 2 μl Cecropin B (Sigma) at 100 μg/ml, 10 μg/ml, 1 μg/ml and 0.1 μg/ml (FIG. 10). Haemolymph taken after 48h of induction by P. aeruginosa produced an antimicrobial plaque of 5 mm diameter—greater than that produced by the 10 μg/ml cecropin standard (4.25 mm) but smaller than the 100 μg/ml standard (8 mm).

Conclusion—Anti-bacterial activity was found in L. sericata haemolymph after induction by exposure to P. aeruginosa during growth.

### REFERENCES

O'Toole, G. A. and Kolter, R. (1999) Initiation of biofilm formation in Pseudomonas fluorescens WCS365 proceeds via multiple, convergent signalling pathways: a genetic analysis. Molecular Microbiology 28: 449-461.
Sherman, R. A. and Tran, J. M. (1995) A simple, sterile food source for rearing the larvae of Lucilia sericata (Diptera; Calliphoridae). Medical and Veterinary Entomology 9: 393-398.
Thomas, S., Andrews, A. M., Hay, N. P. and Bourgoise, S. (1999) The anti-microbial activity of maggot secretions: results of a preliminary study. J. Tissue Viability 9: 127-132.
Hoffmann, J. A., Kefatos, F. C., Janeway, C. A. and Ezekowitz, R. A. B. (1999), Phylogenetic perspectives in innate immunity, Science; 284; 1313-1318.

1. A method of treating a surface populated by a bacteria capable of producing a biofilm which comprises contacting the surface with a substance having N-acyl homoserine lactone degradant activity obtained from the secretions/excretions of Lucilia sericata.

2. A method according to claim 1, wherein the surface is selected from metal surfaces, glass surfaces and the surfaces of plastics materials.

3. A method according to claim 1 or claim 2, wherein the surface is the surface of a medical device or implant.

4. A method according to claim 1, wherein the surface is a wound surface.

5. A method according to any one of claims 1 to 4, wherein the bacteria capable of producing a biofilm is Pseudomonas aeruginosa or Staphylococcus aureus.

6. A method according to any one of claims 1 to 5, wherein the substance is provided in a composition which additionally comprises one or more antibiotic compound.

7. A method according to claim 6, wherein the antibiotic compound is tetracycline.

8. An antimicrobial composition comprising secretions/excretions isolated from Lucilia sericata or analogues thereof and one or more antibiotic compound.

9. A composition according to claim 8, wherein the antibiotic compound is tetracycline.

10. An antimicrobial composition comprising, as an active component, a substance having N-acyl homoserine lactone-degradant activity isolated from secretions/excretions obtained from Lucilia sericata or analogues thereof together with a carrier or vehicle.

11. An antimicrobial composition comprising, as an active component, a serine proteinase isolated from secretion/excretions obtained from Lucilia sericata or analogues thereof together with a carrier or vehicle.

12. An antimicrobial composition comprising, as an active component, a m glycosidase isolated from secretions/excretions obtained from Lucilia sericata or analogues thereof together with a carrier or vehicle.

13. An antimicrobial composition comprising, as an active component, a substance having cecropin-like activity isolated from secretions/excretions obtained from Lucilia sericata or analogues thereof together with a carrier or vehicle.

14. A composition according to any one of claims 10 to 13 which additionally comprises one or more antibiotic compound.

15. A composition according to claim 14, wherein the antibiotic compound is tetracycline.

16. An antimicrobial composition comprising cell-free haemolymph obtained from Lucilia sericata larvae grown in the presence of Pseudomonas aeruginosa, or one or more active constituent of said haemolymph or a synthetic analogue of such constituent.

17. A wound dressing comprising a composition according to any one of claims 8 to 16 together with a carrier.
